(19) United States
(12) Patent Application Publication
(10) Pub. No.: US 2026/0306039 A1
(43) Pub. Date: Oct. 8, 2026

(54) SYSTEMS AND METHODS FOR EXTRACTING VERTEBRAL BODIES FROM TWO-DIMENSIONAL IMAGE DATA AND CLASSIFYING FRACTURE

(71) Applicant: University of Washington, Seattle, WA (US)

(72) Inventors: Nathan M. Cross, Seattle, WA (US); Jeffrey Jarvik, Seattle, WA (US); David R. Haynor, Seattle, WA (US); Gang Luo, Seattle, WA (US); Sandra K. Johnston, Seattle, WA (US); Qifei Dong, Seattle, WA (US); Jonathan Renslo, Seattle, WA (US); Charles Davis, Seattle, WA (US); Brian Chang, Seattle, WA (US); Jessica Perry, Seattle, WA (US)

(21) Appl. No.: 19/480,345

(22) PCT Filed: May 1, 2024
(86) PCT No.: PCT/US2024/027297

§ 371 Date: Oct. 30, 2025

Related U.S. Application Data
(60) Provisional application No. 63/463,823, filed on May 3, 2023.

(51) Int. Cl. A61B 6/50; A61B 6/00; G06T 7/00; G06V 10/764; G16H 50/20

(52) U.S. Cl. CPC A61B 6/505; A61B 6/5217; G06T 7/0012; G06V 10/764; G16H 50/20; G06T 2207/10116; G06T 2207/20081; G06T 2207/30012; G06V 2201/033

(57) ABSTRACT

Examples described herein include computer systems used to classify a condition, such as fracture, from two-dimensional image data. Examples may find use in screening radiographs for fracture, such as osteoporotic compression fracture (OCF). To screen for fracture, examples described herein may extract image data associated with one or more individual vertebral bodies from the image data of a radiograph. The portion of image data associated with an individual vertebral body may be scored using one or more machine learning models. The scores for individual vertebral bodies and/or clinical covariate data may be combined to classify the radiograph.

### CROSS-REFERENCE TO RELATED APPLICATIONS

This application claims the benefit under 35 U.S.C. § 119 of the earlier filing date of U.S. Provisional Application Ser. No. 63/463,823 filed May 3, 2023, the entire contents of which are hereby incorporated by reference in their entirety for any purpose.

### STATEMENT REGARDING RESEARCH & DEVELOPMENT

This invention was made with government support under Grant No. P30AR072572, awarded by the National Institute of Arthritis and Musculoskeletal and Skin Diseases. The government has certain rights in the invention.

### TECHNICAL FIELD

Examples described herein relate generally to image processing. Examples of fracture detection from radiographs are described. Screening of radiographs using artificial intelligence may be provided in some examples.

### BACKGROUND

Analysis of radiographs may be used to diagnose certain conditions, such as fracture. Fracture is a relevant concern for subjects with osteoporosis. Osteoporosis affects roughly 9% of individuals over 50 years old in the United States (US) and 200 million women globally. In developed countries, one out of three individuals will suffer an osteoporotic compression fracture (OCF) in their lifetime. Even one OCF can decrease quality of life and increase risk of mortality. OCFs are indicators of the presence of osteoporosis, and their detection can lead to further evaluation and treatment.

Osteoporosis screening is evidence-based and is endorsed by many organizations, including the US Preventive Services Task Force, but remains underutilized. Between 2004 and 2006, more than ⅔ of women who should have been screened for osteoporosis were not.

### BRIEF SUMMARY

Examples of methods are described herein. In some examples, a method includes extracting, using a first trained machine learning model, image data portions corresponding to respective individual vertebral bodies from a two-dimensional image of at least a portion of a spine, inputting each of the image data portions to a second machine learning model to generate respective scores indicative of a likelihood each of the individual vertebral bodies has a fracture, classifying the spine, using a subject-level classifier based on the scores, into a fracture status.

The example method may also include where the image data portions comprise first image data portions, and where the method further includes extracting, using a third trained machine learning model, second image data portions corresponding to the respective individual vertebral bodies from the two-dimensional image. In some examples, the two-dimensional image is generated by an X-ray system. In some examples, classifying the spine is further based on clinical covariate data. In some examples, the method may include displaying the fracture status overlaid on the two-dimensional image.

Examples of computer readable media are described herein. In some examples, at least one computer readable media is encoded with instructions, which, when executed, cause a system to perform operations including extract, using a first trained machine learning model, image data portions corresponding to respective individual vertebral bodies from a two-dimensional image of at least a portion of a spine, input each of the image data portions to a second machine learning model to generate respective scores indicative of a likelihood each of the individual vertebral bodies has a fracture, and classify the spine, using a subject-level classifier based on the scores, into a fracture status.

Examples of systems are described herein. In some examples, a system includes at least one processor. The system also includes at least one computer readable media encoded with instructions which, when executed by the at least one processor, cause the system to perform operations including extract, using a first trained machine learning model, image data portions corresponding to respective individual vertebral bodies from a two-dimensional image of at least a portion of a spine, input each of the image data portions to a second machine learning model to generate respective scores indicative of a likelihood each of the individual vertebral bodies has a fracture, and classify the spine, using a subject-level classifier based on the scores, into a fracture status.

### BRIEF DESCRIPTION OF THE DRAWINGS

FIG. 1 is a schematic illustration of a system, arranged in accordance with examples described herein.

FIG. 2 is a flowchart illustrating an example method arranged in accordance with examples described herein.

FIG. 3A is a schematic illustration of an example method for scoring vertebral bodies arranged in accordance with examples described herein.

FIG. 3B is a schematic illustration of an example method for scoring vertebral bodies arranged in accordance with examples described herein.

FIG. 3C is a schematic illustration of an example method for scoring vertebral bodies arranged in accordance with examples described herein.

FIG. 4 is a sheet of equations referred to herein.

### DETAILED DESCRIPTION

Certain details are set forth herein to provide an understanding of described embodiments of technology. However, other examples may be practiced without various of these particular details. In some instances, well-known computer system components, circuits, control signals, timing protocols, software operations, anatomical specifics, clinical data, and/or medical conditions have not been shown in detail in order to avoid unnecessarily obscuring the described embodiments. Other embodiments may be utilized, and other changes may be made, without departing from the spirit or scope of the subject matter presented here.

Using bone mineral density as a biomarker of osteoporosis detection has known limitations. Spinal Osteoporotic Compression Fractures (OCFs) can serve as an additional osteoporosis biomarker and are often incidental on chest, spine, or abdominal images and are frequently under-reported, resulting in underdiagnosis and under-treatment. Applying automated OCF screening to imaging studies could result in earlier and more extensive osteoporosis identification and treatment.

Examples described herein include an automated fracture (e.g., OCF) radiograph screening tool. Multiple image processing tools, including multiple artificial intelligence models, analyze medical radiographs, including parts of the spine, to create a summary prediction of whether one or more fractures are present on the radiograph. This prediction of fracture can be used to alert providers or radiologists of the potential presence of fracture and need for further screening for osteoporosis. In addition to running this screening tool on images taken for the express purpose of fracture detection, examples of systems described herein could be used to screen incoming radiographs, imported or acquired at a given medical institution, to look for evidence of osteoporosis and aid in ensuring evidence-based work up and treatment of those patients.

Examples described herein include computer systems used to classify a condition, such as fracture, from two-dimensional image data. Examples may find use in screening radiographs for fracture, such as OCF. To screen for fracture, examples described herein may extract image data associated with one or more individual vertebral bodies from the image data of a two-dimensional image, such as a radiograph. The portion of image data associated with an individual vertebral body may be scored using one or more machine learning models. The scores for individual vertebral bodies and/or clinical covariate data may be combined to classify the radiograph.

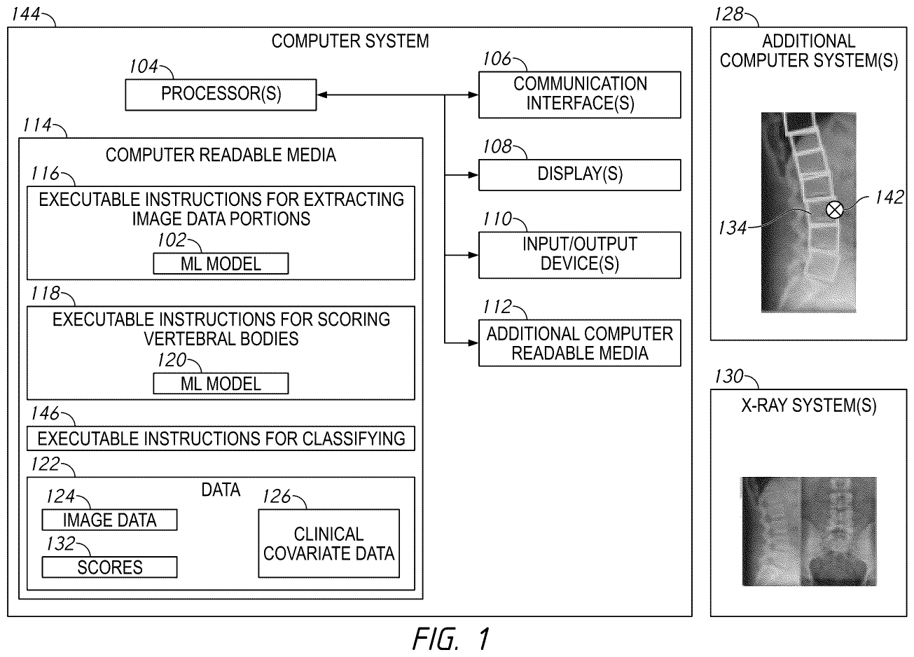

FIG. 1 is a schematic illustration of a system arranged in accordance with examples described herein. The system of FIG. 1 includes computer system 144, additional computer system(s) 128, and X-ray system(s) 130. The computer system 144 may include processor(s) 104, computer readable media 114, communication interface(s) 106, display(s) 108, input/output device(s) 110, and additional computer readable media 112. The computer readable media 114 may include executable instructions for extracting image data portions 116, executable instructions for scoring vertebral bodies 118, executable instructions for classifying 146, and data 122. The executable instructions for extracting image data portions 116 may include machine learning (ML) model 102. The executable instructions for scoring vertebral bodies 118 may include ML model 120. The data 122 may include image data 124, scores 132, and clinical covariate data 126. The additional computer system(s) 128 may display an image including vertebral body 134 and indicator 142.

FIG. 1 is an exemplary system depiction. Additional, fewer, and/or different components may be used in other examples.

Computer systems, such as the computer system 144 of FIG. 1, may include one or more processors, such as the processor(s) 104. Any kind and/or number of processors may be present, including one or more central processing unit(s) (CPUs), graphics processing units (GPUs), other computer processors, processor cores, mobile processors, digital signal processors (DSPs), field programmable gate arrays (FPGAs), application specific integrated circuits (ASICs), microprocessors, controllers and/or processing units configured to execute machine-language instructions and process data.

Computer systems, such as the computer system 144 of FIG. 1, may further include computer readable media, such as computer readable media 114. Any type or kind of media may be present, including memory and/or storage. Examples include read only memory (ROM), random access memory (RAM), solid state drive (SSD), secure digital card (SD card), hard drive, network-attached storage, etc. While a single schematic square is depicted as computer readable media 114 in FIG. 1, any number of memory and/or storage devices may be present, and the executable instructions and/or data shown may be stored on one or more separate devices in some examples. The computer readable media may be in communication (e.g., electrically connected) to the processor(s), such as computer readable media 114 being coupled to processor(s) 104.

The computer readable media may store executable instructions for execution by the processor(s), such as executable instructions for extracting image data portions 116 and/or executable instructions for scoring vertebral bodies 118. The executable instructions for extracting image data portions 116 may be executed by one or more processor(s) 104 to extract portions of image data which depict individual vertebral bodies.

Image data herein may generally refer to data representative of one or more images or portions of images. An example of image data is pixel data, which may include one or more intensity and/or color values per pixel and/or groups of pixels that may together form an image. Examples of images described herein include two-dimensional images, such as radiographs. A radiograph generally refers to images generated using one or more X-ray systems. In the radiograph, denser materials generally absorb more incoming radiation, and accordingly appear lighter in the image than less dense materials. Note that an X-ray generally takes a projection image, generally each position of an image of a radiograph is generally associated with an entire thickness of a sample (e.g., a portion of a subject, such as a human subject). The incident radiation travels through the thickness of the subject. This is in contrast to, for example, computed tomography (CT) images or data which is generally reconstructed as a volume, representing a slice-by-slice image through a subject. Radiographs may be more easily and cheaply obtained than CT data, making systems described herein advantageous for use in screening populations where it may not be feasible to obtain CT data. However, radiographs pose unique challenges to segmentation as they are projection images, and objects can distort and superimpose, obscuring key geometric features. However, their ubiquity in clinical practice, low radiation dose, and relatively low cost, may be advantageous for use in a screening tool. While examples of radiographs have been described, other two-dimensional images and/or data may be used in other examples including, but not limited to, scout images, and/or CT data projected into a 2D image.

In examples described herein, radiographs may include an image of an entire spine and/or portions of a spine. Any subject having a spine may generally be imaged by X-ray systems described herein to generate a radiograph including, but not limited to, one or more humans (e.g., elderly subject, child, infant, fetus, cadaver) and/or one or more animals (e.g., dog, cat).

The executable instructions for extracting image data portions 116 may include instructions for accessing image data pertaining to a radiograph. Image data portions may be extracted from the radiograph which pertain to individual vertebral bodies. In this manner, the computer readable media 114 may analyze image data of radiographs and extract portions of image data which are representative of individual vertebral bodies. Extracting generally refers to identifying the image data associated with one or more individual vertebral bodies. The extracted image data portion may in some examples be stored separately and/or data specifying the location of the image data portion in radiograph image data may be stored. The image data portion may be associated with, for example, the bone of an individual vertebral body. In some examples, the image data portion may include an amount of border around the bone of an individual vertebral body.

Accordingly, the executable instructions for extracting image data portions 116 may include executable instructions for implementing an ML model, such as ML model 102 of FIG. 1. The ML model 102 may be a model trained to output image data portions corresponding to individual vertebral bodies based on image data associated with a radiograph. In this manner, the image data associated with individual vertebral bodies may be extracted. For example, a section of pixel data corresponding to an individual vertebral body may be identified. Any number of vertebral bodies may be identified. Vertebral bodies identified may include image data associated with one or more cervical vertebrae, thoracic vertebrae, lumbar vertebrae, and/or fused vertebrae. In some examples, thoracic and/or lumbar vertebrae may be identified, because those are the most likely to contain fractures, and/or certain types of fractures. Image data associated with any number of vertebral bodies may be extracted, including 1, 2, 3, 4, 5, 6, 7, 8, 9, 10, 11, 12, 13, 14, 15, 16, 17, 18, 19, 20, 21, or another number of vertebral bodies.

While in some examples, an ML model, such as ML model 102 may be used to extract individual vertebral bodies, in some examples, machine learning may not be used. For example, one or more corner points or other features of vertebral bodies may be annotated, and the executable instructions for extracting image data portions 116 may extract image data portions based on the annotations.

In some examples, the executable instructions for extracting image data portions 116 may include multiple ML models, with each ML model trained to extract image data portions associated with individual vertebral bodies in a different manner. While not shown in FIG. 1 explicitly, the executable instructions for extracting image data portions 116 may include instructions for implementing a second ML model. The additional ML model may also extract image data portions from a radiograph corresponding to individual vertebral bodies, however the additional ML model may do so utilizing a different ML technique and/or training than ML model 102. The executable instructions for extracting image data portions 116 may include instructions for combining the image data portions extracted using the ML model 102 with image data portions extracted using an additional ML model in order to obtain final extracted image data portions. Utilizing multiple ML models to extract the image data portions may allow the final extracted image data portions to be more accurate as to portions that correspond to individual vertebral bodies. The final extracted image data portions may be used by the executable instructions for scoring vertebral bodies 118 to score the individual vertebral bodies.

In this manner, the executable instructions for extracting image data portions 116 may be used to identify image data portions associated with individual vertebral bodies. In some examples, the executable instructions for extracting image data portions 116 may include instructions for depicting those portions of image data on an image of the radiograph. For example, the image data portions may be shaded using a different color or intensity, and/or a box or other shape may be used to outline the individual image data portions.

By extracting image data portions associated with individual vertebral bodies, each individual vertebral body (and/or groups of vertebral bodies) may be independently analyzed as described herein. For example, each individual vertebral body may be analyzed for a likelihood of fracture and/or other status. By analyzing image data portions associated with individual vertebral bodies (and/or groups of vertebral bodies) independently, the reliability and/or accuracy of the subsequent analysis may be improved relative to examples in which the image data of a larger portion of the radiograph is analyzed (e.g., image data associated with an entire spinal radiograph).

Examples of systems described herein may include executable instructions for scoring vertebral bodies, such as executable instructions for scoring vertebral bodies 118 of FIG. 1. The executable instructions for scoring vertebral bodies 118 may analyze the extracted portion of image data associated with each individual vertebral body. For example, the executable instructions for scoring vertebral bodies 118 may operate on some or all of the image data portions extracted using executable instructions for extracting image data portions 116. For each image data portion, the executable instructions for scoring vertebral bodies 118 may generate a score indicative of the likelihood of a particular condition. Systems described herein may score for any of a variety of conditions, such as fracture. In some examples, the executable instructions for scoring vertebral bodies 118 may provide a score for a particular kind of fracture, such as an osteoporotic fracture. An osteoporotic fracture refers generally to a fracture which may occur in a subject with osteoporosis.

Prior to and/or during analysis of image data portions in accordance with the executable instructions for scoring vertebral bodies 118, one or more image pre-processing operations may be performed. The computer readable media 114 may include executable instructions for pre-processing (not shown in FIG. 1). The pre-processing operations may include augmenting brightness and/or contrast. Other pre-processing operations may include adding and/or subtracting noise from the image data portions. In some examples, pre-processing may include flipping the image to achieve a particular orientation of the spine (e.g., left or right). In some examples, pre-processing may include inverting the greyscale and/or color of the image data. For example, the grayscale may be inverted if the bones are darker than the background. Additional or different resizing, scaling, augmenting, and/or normalizing may be used in some examples. Pre-processing may be performed to obtain image data in a standard format for providing to a next analysis operation (e.g., segmentation).

The executable instructions for scoring vertebral bodies 118 may include instructions for implementing an ML model, such as ML model 120. The ML model 120 may be trained to output a likelihood of a condition (e.g., fracture) based on input image data. The ML model 120 may be trained for a particular condition. For example, the ML model 120 may be trained to identify a likelihood of fracture in image data associated with an individual vertebral body. The ML model 120 may be trained to identify likelihood of a specific type of fracture, such as an osteoporotic fracture in some examples. In some examples, multiple ML models may be included in executable instructions for scoring vertebral bodies 118 and a signal indicating a selection of a condition may be provided to the computer readable media 114. The ML model used may be selected based on the condition selected (e.g., osteoporotic fracture in some examples, other types of fracture in other examples, and/or other conditions in other examples).

The ML model 120 may be trained in any of a variety of ways, including using supervised or unsupervised learning. In some examples, the ML model 120 may be trained on a set of training data. The training data may include image data for spinal radiographs in some examples. The training data may include expert classifications of the radiographs and/or of individual vertebral bodies in the radiographs as to a likelihood that each contains an image of a fracture. In some examples, the training data includes radiographs taken at a variety of locations and/or with a variety of X-ray systems and/or a variety of X-ray system operators and/or from a specific subject population. In some examples, the training data may be specific to a location and/or to an X-ray system, to a type of X-ray system, and/or to an X-ray system operator and/or to a specific patient population (e.g., subjects having osteoarthritis). In some examples, multiple ML models may be provided, each trained on training data specific to a location (e.g., a healthcare facility), an X-ray system, and/or an X-ray system operator and/or a patient population. In some examples, a selection signal may be provided to the computer system 144 indicative of a location (e.g., a healthcare facility), an X-ray system and/or type of X-ray system, and/or an X-ray system operator, and/or a patient population, and an ML model may be selected and utilized in executable instructions for scoring vertebral bodies 118 based on the selection signal.

In some examples, although not explicitly shown in FIG. 1, multiple ML models may be used to score image data portions described herein. Accordingly, different types and/or training sets may be used to provide different ML models. Multiple ML models may be used to independently score the image data portions. The output of multiple ML models may be combined in accordance with the executable instructions for scoring vertebral bodies 118 to provide a final score for an individual vertebral body. For example, ensemble averaging and/or ensemble voting may be used to combine outputs from multiple ML models to obtain the final score.

The score output using the executable instructions for scoring vertebral bodies 118 may be, for example, a percentage or other normalized value. A score may be provided for each individual vertebral body (and/or groups of vertebral bodies in some examples). In this manner, for a single radiograph depicting multiple individual vertebral bodies, the executable instructions for extracting image data portions 116 may extract image data portions relating to individual vertebral bodies, and the executable instructions for scoring vertebral bodies 118 may score each of those image data portions as to their likelihood of depicting a fracture or other condition. Note that the identification of fracture may be complex in some examples.

Examples of systems described herein may classify image data, such as image data of one or more radiographs. Accordingly, computer system 144 may include executable instructions for classifying 146. The executable instructions for classifying 146 may also be referred to as a classifier. The classifier may be referred to as a subject-level classifier. Accordingly, the executable instructions for classifying 146 may be used to classify one or more radiographs on a subject level (e.g., for a particular subject such as a patient, human, and/or animal). The executable instructions for classifying 146 may be executed by the processor(s) 104 to receive one or more scores of individual image data portions corresponding to vertebral bodies. Based on the scores, the processor(s) 104 may execute the executable instructions for classifying 146 to classify a radiograph as having a particular condition—e.g., a fracture. The classifier may utilize a variety of criteria to classify a radiograph. For example, a combination of the scores of individual vertebral bodies in the radiograph may be used to classify the radiograph. In some examples, the executable instructions for classifying 146 may classify a radiograph as having a condition if a threshold number or more of the vertebral bodies have over a threshold likelihood of having the condition (e.g., fracture). In some examples, the executable instructions for classifying 146 may classify a radiograph as having a condition if a greater number of the vertebral bodies have over a lower threshold likelihood of having the condition. So, as an example, the classifier may classify the radiograph as having a condition if one of the vertebral bodies has a greater than 90 percent likelihood of having the condition. Other numbers of vertebral bodies and likelihoods may be used in other examples. However, in some examples, the condition may not be clear, but the presence of multiple less certain vertebral bodies having the condition may be used to classify the radiograph. For example, the executable instructions for classifying 146 may classify a radiograph as having the condition when three or more vertebral bodies have a 50 percent likelihood of having the condition. Other numbers of vertebral bodies and likelihoods may be used in other examples. In some examples, logistic regression (LR) and/or generalized additive models (GAM) may be utilized to combine individual vertebral scores. In some examples, a GAM utilizing a number of top vertebral facture scores (e.g., top three in one example) may be used.

In some examples, the executable instructions for classifying 146 may include instructions for utilizing additional data, in addition to the scores of the image data portions, to classify a radiograph. For example, clinical covariance data, such as clinical covariate data 126 may be used in some examples to classify or influence classification of the radiograph. The clinical covariate data 126 may include generally structured or unstructured data. The clinical covariate data 126 may include data from an electronic medical record. Examples of clinical covariate data 126 may include one or more of age, gender, race, ethnicity, demographics, and body mass index (BMI); current and historical diagnoses (e.g., using International Classification of Disease (ICD) code and/or hierarchical condition categories (HCC)), medications, and diagnoses and exam findings extracted from free text clinical and/or procedure notes; or other elements captured or stored in an electronic medical record. Other clinical covariate data may be used in other examples.

The computer readable media 114 and/or other computer readable media accessible to the computer system 144 may store data, such as data 122. The data may be generated by the computer system 144, received by the computer system 144, and/or accessed by the computer system 144. For example, image data 124 may include pixel data representing one or more radiographs. The image data 124 may include pixel data representing one or more image data portions extracted in accordance with executable instructions for extracting image data portions 116. The scores 132 may include one or more scores calculated or otherwise determined in accordance with the executable instructions for scoring vertebral bodies 118. The image data 124 and/or scores 132 may be accessed and/or otherwise used by the executable instructions for classifying 146. The clinical covariate data 126 may be accessed and/or otherwise used by the executable instructions for classifying 146. The clinical covariate data 126 may be obtained from one or more other computing systems in some examples, such as a computing system hosting electronic medical records.

The computer system of FIG. 1 may include additional components, not all of which are necessarily depicted in FIG. 1. Examples of additional components may include one or more communication interface(s), such as communication interface(s) 106. The communication interface(s) 106 may include a WiFi, Ethernet, Bluetooth, network interface, cellular interface and/or other communications interface(s). The computer system 144 may include one or more display(s), such as display(s) 108. The display(s) 108 may display one or more radiographs, images of one or more image data portions described herein, radiographs overlaid with indications of the individual vertebral bodies and/or scores for the individual vertebral bodies and/or classification of the radiograph. The display(s) 108 may display classifications of one or more radiographs and/or scores of one or more individual vertebral bodies.

The computer system 144 may include one or more input and/or output devices, such as input/output device(s) 110 including, but not limited to, one or more touchscreens, mice, keyboards, and/or cameras. The computer system may include and/or be in communication with additional computer readable media, such as additional computer readable media 112. The additional computer readable media 112 may include, for example, an electronic medical record or other source of clinical covariate data 126.

Examples of systems described herein may include one or more additional computer systems, such as additional computer system(s) 128 of FIG. 1. The additional computer system(s) 128 may, for example, be a computer system for use by one or more clinicians. In some examples, the additional computer system(s) 128 may be in communication, wired or wireless, with the computer system 144 and/or with data 122. The additional computer system(s) 128 may be used to display classifications, scores, image data portions, and/or radiographs described herein. In the example of FIG. 1, the additional computer system(s) 128 is depicted as including a display which is displaying a radiograph of a spine showing boxes around the individual vertebral bodies, including vertebral body 134, whose image data portions had been extracted in accordance with executable instructions for extracting image data portions 116. The radiograph further is displayed with indicator 142. The indicator 142 is displayed on the radiograph to indicate that the vertebral body 134 has a likelihood of fracture greater than a threshold. In some examples, an indicator may be provided for the radiograph as a whole indicating a classification determined in accordance with executable instructions for classifying 146.

One or more clinicians may accordingly view classifications, radiographs, image data portions, and/or individual vertebral bodies described herein. Based on the classifications and/or image data portions, one or more clinicians may provide an input to the additional computer system(s) 128 to clinically determine that a particular subject has a condition (e.g., fracture). In some examples, the clinician may agree with the output of the computer system 144 in classifying the radiograph. But in other examples, the clinician may not agree and may classify the radiograph differently than the computer system 144. Generally, however, in examples described herein, a classification made by the output of the computer system 144 may be accurate and may generally cause the clinician to agree with the classification. Radiographs being classified by the computer system 144 as having the condition may be prioritized for review by a clinician. For example, the additional computer system(s) 128 may present the clinician first with the radiographs having been classified by the computer system 144 as having the condition. Radiographs classified by the computer system 144 as not having the condition may be reviewed by a clinician after review of the radiographs classified by the computer system 144 as having the condition.

In some examples, the classifications, radiographs, image data portions, and/or individual vertebral bodies may be viewed to diagnose a condition, such as osteoporosis. For example, radiographs classified as containing a fracture may be provided to another computing system for use in screening the subject of the radiograph for osteoporosis or another diagnosis. Accordingly, clinicians viewing outputs of systems described herein may diagnose and/or treat a condition in part based on the classification suggested by the system.

Examples of systems described herein may include and/or communicate with one or more X-ray systems, such as X-ray system(s) 130 of FIG. 1. The X-ray system(s) 130 may be in communication, wired or wireless, with the computer system 144 in some examples. In some examples, the computer system 144 may be integrated into one or more computer systems used to control or otherwise interact with the X-ray system(s) 130. Any of a variety of X-ray systems may be used. Generally, the X-ray system(s) 130 may be used to obtain one or more image data for radiographs of a subject. In the example of FIG. 1, spinal radiographs of a subject obtained by the X-ray system(s) 130 are depicted. The image data for the radiograph(s) generated by the X-ray system(s) 130 may be provided to the computer system 144, e.g., as image data 124.

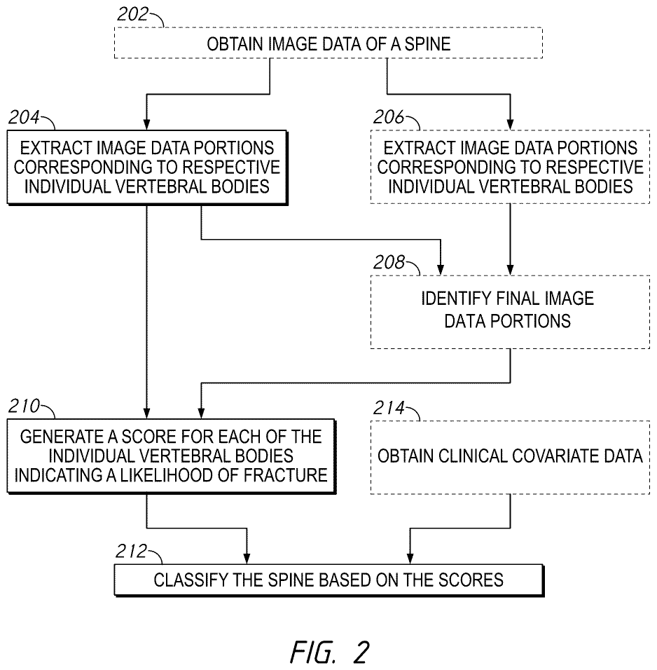

FIG. 2 is a flowchart illustrating an example method arranged in accordance with examples described herein.

The method of FIG. 2 includes block 202 which recites “obtain image data of a spine.” The block 202 may be followed by block 204 which recites “extract image data portions corresponding to respective individual vertebral bodies.” The block 202 may be followed by block 206 in some examples. The block 206 recites “extract image data portions corresponding to respective individual vertebral bodies.” The block 204 and the block 206 may be followed by block 208 in some examples. The block 208 recites “identify image data portions.” The block 204 and/or the block 208 may be followed by block 210 which recites “generate a score for each of the individual vertebral bodies indicating a likelihood of fracture.” Block 214 may be present. The block 214 recites “obtain clinical covariate data.” The block 210 and/or the block 214 may be followed by block 212. The block 212 recites “classify the spine based on the scores.”

The flowchart of FIG. 2 is exemplary. Additional, fewer, and/or different blocks may be included in other examples. The method of FIG. 2 may be implemented using computer systems described herein, such as the computer system 144 of FIG. 1. For example, the computer readable media 114 of FIG. 1 may include instructions for, when executed by the processor(s) 104 of FIG. 1, performing some or all of the operations described with respect to FIG. 2.

Examples of methods described herein may obtain image data of a spine, such as in block 202 of FIG. 2. The image data may be generated using one or more X-ray systems, such as the X-ray system(s) 130 of FIG. 1. Accordingly, the image data may be image data of a radiograph. In some examples, other two-dimensional image data of a spine may be obtained, such as by projecting CT data into a two-dimensional representation. The image data may be image data of all or a portion of a spine of a subject. The image data may be provided in block 202 to a computer system from another computer system. For example, image data may be generated by X-ray system(s) 130 of FIG. 1 and provided to the computer system 144 as image data 124. In some examples, block 202 refers to obtaining image data by accessing the image data, such as by processor(s) 104 accessing the image data 124.

Examples of methods described herein may extract image data portions corresponding to respective individual vertebral bodies, such as in block 204. For example, the executable instructions for extracting image data portions 116 may include instructions for analyzing the image data of a radiograph and extracting image data portions corresponding to one or more individual vertebral bodies. The executable instructions for extracting image data portions 116 may include instructions for implementing an ML model 102. Accordingly, an ML technique may be used in block 204 to extract image data portions corresponding to individual vertebral bodies. The extracted image data portions may be extracted by identifying the positions of pixel data within image data of a radiograph that correspond to the individual vertebral bodies. In some examples, the extracted image data portions may be stored separately—e.g., image data portions for individual vertebral bodies may be stored separately and may be separately accessed, e.g., by computer system 144 in accordance with executable instructions for scoring vertebral bodies 118.

In some examples, another technique may be used to extract image data portions corresponding to individual vertebral bodies, such as in block 206. For example, the executable instructions for extracting image data portions 116 may include instructions for analyzing the image data of a radiograph and extracting image data portions corresponding to one or more individual vertebral bodies in another manner. The executable instructions for extracting image data portions 116 may include instructions for implementing an additional machine learning model in addition to ML model 102. Accordingly, a different ML technique may be used in block 206 to extract image data portions corresponding to individual vertebral bodies. The extracted image data portions may be extracted by identifying the positions of pixel data within image data of a radiograph that correspond to the individual vertebral bodies. In some examples, the extracted image data portions may be stored separately—e.g., image data portions for individual vertebral bodies may be stored separately and may be separately accessed, e.g., by computer system 144 in accordance with executable instructions for scoring vertebral bodies 118.

In some examples, image data portions identified by multiple techniques may be used to make a final identification of image data portions of the individual vertebral bodies, such as in block 208. The image data portions may be combined using computer system 144 in accordance with the executable instructions for extracting image data portions 116. In some examples, the executable instructions for extracting image data portions 116 may include instructions for combining the image data portions from block 204 and block 206. In some examples, combining the image data portions may include retaining all image data identified by either block 204 and/or block 206. In some examples, combining the image data portions may include retaining only image data identified by multiple techniques, e.g., in both block 204 and block 206. In some examples, one of the techniques may be weighted in making the combination of block 208. In some examples, the final image data portions may be identified by choosing one of the methods from bock 204 and/or block 206.

Examples of methods described herein may generate a score for each of the individual vertebral bodies. The score may indicate a likelihood of fracture being present in the radiograph of the individual vertebral body. The block 210 may be implemented using computer system 144 of FIG. 1 in accordance with executable instructions for scoring vertebral bodies 118. The block 210 may utilize the image data portions extracted in block 204 and/or output from a combination in block 208. In this manner, in block 210, multiple scores may be generated and stored, for example, as scores 132 of FIG. 1.

Examples of methods described herein may include obtaining clinical covariate data, such as in block 214. The clinical covariate data may be obtained, for example, using computer system 144 to communicate with one or more additional computer systems, such as additional computer system(s) 128. In some examples, the clinical covariate data may be obtained by accessing stored covariate data, such as clinical covariate data 126 of FIG. 1.

Examples of methods described herein may classify a spine, such as in block 212, based on scores calculated in accordance with methods described herein. For example, the computer system 144 may be used to classify image data of a spine in accordance with the executable instructions for classifying 146. The image data may be classified as having fracture or not having fracture. In some examples, other conditions may be classified.

In this manner, image data of one or more radiographs, portions of image data corresponding to individual vertebral bodies, scores for individual vertebral bodies, and/or classifications of spines, may be generated and/or stored by systems described herein, such as in data 122 of FIG. 1. This data may be used to display information and/or to prioritize information for display to one or more clinicians. For example, a radiograph may be displayed together with indications of the location and shape of individual vertebral bodies in the radiograph. The radiograph may be displayed together with one or more indicators of individual vertebral bodies likely to contain a fracture. The radiograph may be displayed together with an indication of the classification of the radiograph. A clinician may use this information to inform their diagnosis and/or treatment of a subject.

Examples of systems described herein may utilize one or more ML models to extract image data portions of radiograph image data. The extracted image data portions may correspond to individual vertebral bodies. In some examples, ensemble techniques may be utilized to combine outputs of convolutional deep neural network segmentation methods to extract image data portions corresponding to individual vertebral bodies. The ML models described for extracting image data portions may be used, for example, to implement ML model 102 of FIG. 1 and/or additional ML models included in executable instructions for extracting image data portions 116.

In some examples, a training dataset may be used to train one or more ML models to extract image data portions corresponding to individual vertebral bodies. In some examples, a training dataset of thousands (e.g., 5,994) of subjects may be used from multiple (e.g., six) clinical sites. Ground truth annotations may be established, in some examples utilizing a semi-automated tool (e.g., SpineAnalyzer and/or Aptasia Medical) to mark edges and/or features of individual vertebral bodies. Generally, radiographs in the training dataset may include lateral thoracic and lumbar radiographs. On thoracic projections, vertebral bodies may be annotated from T5 to T12 or L1 in some examples. On lumbar projections, T12 or L1 to L3 or L4 may be annotated in some examples. Many vertebral bodies (VBs) (e.g., T3, L5) may be visible on radiographs but not annotated in some examples. Accordingly, training datasets may utilize thoracic and/or lumbar VBs.

In some examples, one or both of the convolutional deep neural network segmentation models U-Net and Mask-RCNN may be trained to implement the ML model 102. U-Net is a convolutional neural network which may be used for semantic segmentation. Examples of U-Net are described, for example, in Olaf Ronneberger, Philipp Fischer, & Thomas Brox, U-Net: Convolutional Networks for Biomedical Image Segmentation, Medical Image Computing and Computer-Assisted Intervention—MICCAI 2015 (eds. Navab, N., Hornegger, J., Wells, W. M. & Frangi, A. F.) 234-241 (Springer International Publishing, 2015), which publication is hereby incorporated by reference herein in its entirety for any purpose. Mask-RCNN may generally be suited for object detection tasks. Examples of Mask-RCNN are described, for example, in Kaiming He, Georgia Gkioxari, Piotr Dollár, and Ross Girshick, Mask R-CNN, arXiv (Jan. 24, 2018), doi: 10.48550/arXiv.1703.06870, which publication is hereby incorporated by reference herein in its entirety for any purpose. In some examples, the output of the U-Net model and the Mask-RCNN model may be combined to generate an output of the executable instructions for extracting image data portions 116.

Pre-processing may be performed in some examples on the radiograph image data. For example, Digital Imaging and Communications in Medicine (DICOM) radiograph images (e.g., one or more DICOM files) may be converted to 16-bit tag image files (TIFs). Each TIF may be downsampled to 512×512 pixels with zero-padding to keep the original aspect ratio. For Mask-RCNN, TIF files may be converted to 8-bit for model training. For U-Net, images may be preprocessed using contrast limited adaptive histogram equalization (CLAHE).

U-Net may be trained in some examples de novo to detect VBs collectively from background (e.g., two class semantic segmentation), producing a per-pixel probability map of VB vs. background classes. Mask-RCNN may be trained to detect individual VBs from lateral radiographs (e.g., instance segmentation). Transfer learning may be applied by importing pre-trained weights, then fine-tuned on a spinal radiograph dataset.

For U-Net, the model output may be binarized, and individual VBs may be distinguished by considering separate contiguous patches filtered for size, ultimately producing centroid coordinates for individual VBs. For Mask-RCNN, centroid coordinates may be calculated from each segmentation mask, which may be re-calculated based on the original DICOM dimensions.

An ensemble model may be formed by pooling predictions and eliminating duplicates: the distance between inference centroids from U-Net and Mask-RCNN may be calculated, and those within half of the average endplate width per radiograph may be considered matches, e.g., the same VB. Mask-RCNN inference centroids may be pooled with the U-Net inference centroids not labeled as matches on a per radiograph basis.

Generally, a variety of anatomic structures and boundaries may present challenges to ML models, such as Mask-RCNN, in detecting VBs on some radiographs. Features which may be associated with off-column detection include overlap of VBs with ribs, bowel gas, spinous processes, and the diaphragm, along with image corruptions including “Mach lines” and corruption of annotations (e.g., annotations flipped horizontally relative to base image). With respect to “gap” VBs, features which may affect extraction include reversed contrast and underexposure, transition in density, parallax, and/or diffuse idiopathic skeletal hyperostosis.

Both U-Net and Mask-RCNN may demonstrate high performance in detecting VBs on lateral radiographs. Combining the inferences in an ensemble method may generally increase the number of correctly identified VBs and the sensitivity. This may provide better data for the fracture classification model downstream.

Accordingly, using an ensemble method to combine the inferences from two independent machine learning models (e.g., segmentation models) in detecting vertebral bodies on lateral radiographs of the thoracic and lumbar spine, may generally increase the number of vertebral bodies detected and the sensitivity, while maintaining high overall performance.

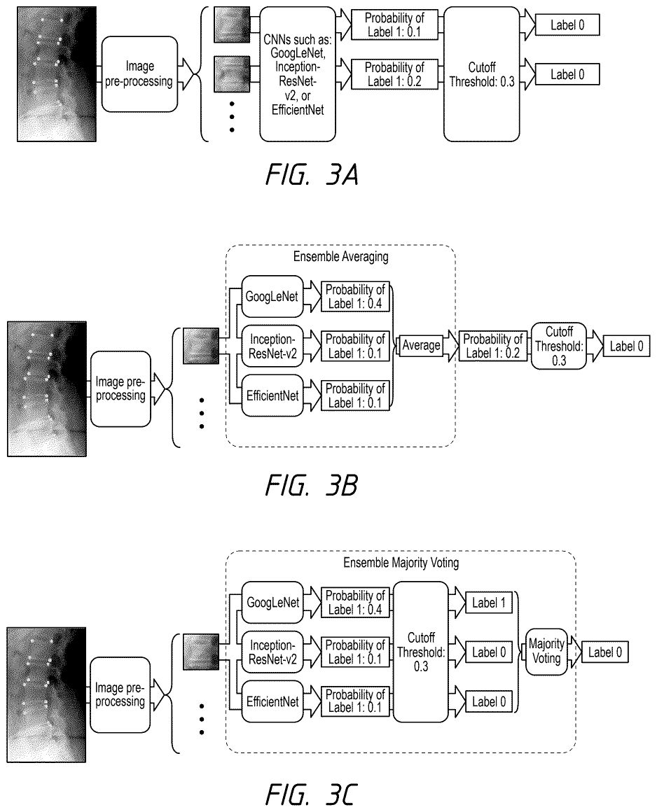

Examples of systems described herein may utilize one or more machine learning models to provide a score of image data portions corresponding to individual vertebral bodies. For example, the ML model 120 of FIG. 1 may be used to score individual vertebral bodies. Examples of scoring are discussed herein. Some examples of classifying each individual vertebral body is described, such as with reference to FIG. 3A, FIG. 3B, and FIG. 3C. In some examples, scoring vertebral bodies may include classifying vertebral bodies into one or more states (e.g., fracture versus no fracture). In some examples, however, a classification may not be performed until a subject-level classification is performed of an entire spine and/or group of vertebral bodies is classified using the scores of individual vertebral bodies.

FIG. 3A, FIG. 3B, and FIG. 3C are schematic illustrations of example methods for scoring vertebral bodies arranged in accordance with examples described herein. The examples of FIG. 3A, FIG. 3B, and FIG. 3C may be implemented using systems described herein, such as the computer system 144 of FIG. 1. The scoring and/or classification described with reference to FIG. 3A, FIG. 3B, and FIG. 3C may be performed, for example by the processor(s) 104 executing executable instructions for scoring vertebral bodies 118. The executable instructions for scoring vertebral bodies 118 may accordingly include instructions for performing the operations described with reference to FIG. 3A, FIG. 3B, and FIG. 3C.

In some examples, a training dataset on the order of thousands (e.g., 1,790 in some examples, 5,994 in some examples) of subjects having on the order of tens of thousands (e.g., 15,050) vertebral bodies may be used to train a machine learning model used to extract image data portions corresponding to individual vertebral bodies. Such a trained machine learning model may be used, for example, to implement ML model 120 which may be used to score vertebral bodies (e.g., to determine a likelihood of fracture in an individual vertebral body). The data may be provided from multiple clinical sites and/or from a single clinical site.

Individual vertebral bodies may be extracted from the training set using ML models described herein. In some examples, individual vertebral bodies may be extracted based on annotations (e.g., expert annotations of corners or other markers for the individual vertebral bodies). The training images may be annotated by one or more reviewers to indicate whether fracture or other condition is present in the image. In some examples, an OCF severity of each individual vertebral bodies may be annotated using a modified-2 algorithm-based qualitative (m2ABQ) criteria. Other criteria may be used to annotate training images in other examples. In some examples, multiple reviewers may annotate each individual vertebral bodies, and the results combined and/or compared to achieve and/or select consensus.

Classification systems and radiologists may struggle to accurately classify mild or subtle fractures often confounded by parallax artifact, remote traumatic injuries, and/or congenital variations. Examples described herein may utilize a binary classification, defined as highly probable fracture versus normal or questionable fracture. Examples described herein may dichotomize the m2ABQ categories: “label 0” representing normal/non-osteoporotic deformity/mild or questionable fracture versus “label 1” representing moderate or severe fracture. Other dichotomies may be used in other examples.

The extracted image data corresponding to individual vertebral bodies and the corresponding annotations of fracture determination may be used to train an ML model to score subsequent image data portions associated with individual vertebral bodies.

Examples of ML models which may be trained on the training data include convolutional neural network models (CNNs) such as GoogLeNet, Inception-ResNet-v2, EfficientNet, and ensemble algorithms. Other models may be used in other examples. Ensemble algorithms may be created using an ensemble averaging algorithm and an ensemble voting algorithm. The ML models may output a probability of fracture for each individual vertebral body.

In some examples, a model may be initially trained on one training data set (e.g., a data set including data from multiple clinical sites) and then fine-tuned on another training data set (e.g., a data set from a specific clinical site).

The examples of threshold and probabilities shown and described with reference to FIGS. 3A, 3B, and 3C are exemplary only. The specific thresholds and probabilities may vary and may be tuned based on the training process and neural network (e.g., CNN) used.

In the example of FIG. 3A, an ML model may be trained and used to calculate a probability of fracture for each individual vertebral body. A radiograph of a portion of a spine is depicted in FIG. 3A, together with annotations of features on individual vertebral bodies. In the example of FIG. 3A, the corners of each individual vertebral body have been annotated and are used to extract the image data portions pertaining to the individual vertebral bodies. Image pre-processing may be performed to adjust and/or standardize various aspects of the image including contrast and/or noise. An ML model may be used to score each individual vertebral body. In the example of FIG. 3A, a GoogLeNet, Inception-ResNet-v2, or EfficientNet ML model may be used to score image data portions corresponding to the individual vertebral bodies. In the example of FIG. 3A, the ML model may score one vertebral body as having a probability of fracture of 0.1 and a second vertebral body as having a probability of fracture of 0.2. In the example of FIG. 3A, a classifier is used with a threshold of 0.3 to classify between fracture (e.g., Label 1) and no fracture (e.g., Label 0). Accordingly, in the example of FIG. 3A, both scored VBs are classified by the classifier as Label 0 because their score (e.g., 0.1) is below the cutoff threshold of 0.3. These trained models may be used, for example, to implement ML model 120 of FIG. 1.

In one example, GoogLeNet was used to predict the presence of OCF for each or a plurality of VBs (e.g., label 1 vs. label 0). This neural network was built using Python 3.7.6, TensorFlow 2.2.0 and TFSlim 1.2.0. GoogLeNet was used in an architecture with one backbone network and two auxiliary networks. Transfer learning was used. In transfer learning, deep learning models pre-trained on large general-purpose imaging datasets are generally used as a starting point for training the model for another task, in this case classifying VBs. TensorFlow Model Garden provided a pre-trained GoogLeNet model implemented in TF-Slim and pre-trained on the ImageNet Large Scale Visual Recognition Challenge 2012 (ILSVRC2012) dataset. The model's output layer was adapted for binary classification to fit OCF classification. Except for the parameters for the output layer, all of the other parameters were initialized from the pre-trained model. The parameters for the output layer and the two auxiliary networks were initialized using He initialization. ILSVRC2012 uses RGB images with three channels. To use the pre-trained weights with the single grayscale channel of each vertebral patch, this channel was copied into the RGB channels. When training GoogLeNet, no layers were frozen. The Adam learning rate optimization was used with a weighted cross entropy loss function which penalizes false positives more heavily than false negatives, and the optimization was iterated over a batch size of 20. Early stopping was used to avoid overfitting. After each epoch, performance was evaluated on the validation set by calculating the AUC-PR. For a highly imbalanced dataset, the AUC-PR is generally a more suitable performance metric than the area under the receiver operating characteristic curve (AUC-ROC) and accuracy. If the AUC-PR did not increase in any of the subsequent 10 epochs, training was terminated. GoogLeNet contains several hyper-parameters. The four hyper-parameters of initial learning rate, learning rate decay, dropout rate, and pos_weight were tuned by random search for 1,500 rounds, with the goal of maximizing the AUC-PR on the validation set. Other hyper-parameters were set to default values. Hyper-parameter tuning was done on two Ubuntu Linux servers concurrently: (1) Xeon E5-2630 with 256 GB memory and four Nvidia Geforce TITAN Xp graphics processing units (GPUs), and (2) Xeon Gold 5215 with 96 GB memory and four Nvidia Geforce 2080 Ti GPUs. The final model was trained and tested on the first server using one GPU.

In the example of FIG. 3B, multiple trained ML models may be used to score image data portions corresponding to individual VBs (e.g., multiple ML models may be included in executable instructions for scoring vertebral bodies 118). For example, a trained GoogLeNet model, a trained Inception-ResNet-v2 model, and a trained EfficientNet model may be used. In one example, the trained GoogLeNet model may provide a probability for one portion of image data of 0.4. The trained Inception-ResNet-v2 model may provide a probability for the same portion of image data of 0.1. The trained EfficientNet model may provide a probability for the same portion of image data of 0.1.

In some examples, the executable instructions for scoring vertebral bodies 118 of FIG. 1 may include executable instructions for combining outputs of multiple ML models. For example, ensemble averaging may be used. Using ensemble averaging in FIG. 3B, the executable instructions for scoring vertebral bodies 118 may include instructions for calculating an average (or weighted average) of the output of multiple ML models. In the example of FIG. 3B, the output probabilities of 0.4, 0.1, and 0.1 are averaged, providing an output score of 0.2.

In the example of FIG. 3B, a classifier is used with a threshold of 0.3 to classify between fracture (e.g., Label 1) and no fracture (e.g., Label 0) for an image data portion corresponding to an individual vertebral body. Accordingly, in the example of FIG. 3B, the scored vertebral body is classified by the classifier as Label 0 because the final averaged score (e.g., 0.2) is below the cutoff threshold of 0.3. These trained models may be used, for example, to implement ML model 120 of FIG. 1.

In the example of FIG. 3C, ensemble majority voting may be used to combine the outputs of multiple ML models. Using ensemble majority voting, a score and/or classification having the most votes (e.g., the highest number of ML models outputting that score) may be selected. In the example of FIG. 3C, as in the example of FIG. 3B, the trained GoogLeNet model may provide a probability for one portion of image data of 0.4. The trained Inception-ResNet-v2 model may provide a probability for the same portion of image data of 0.1. The trained EfficientNet model may provide a probability for the same portion of image data of 0.1. The output probabilities of 0.4, 0.1, and 0.1 are used in accordance with ensemble majority voting instructions to select a probability of 0.1 (and/or classification of no fracture).

Accordingly, in some examples, each individual vertebral body may be classified as having fracture or not having fracture. Each individual vertebral body may be classified in accordance with the executable instructions for scoring vertebral bodies 118 in some examples. A cutoff threshold may be used to classify each individual vertebral body as containing or not containing fracture. For example, if a cutoff threshold of 0.3 was used, then individual VBs having a score of 0.1, 0.2, and/or 0.3 may be classified as not having fracture (e.g., Label 0), while greater scores may be classified as having fracture (e.g., Label 1).

Examples of systems described herein may combine individual vertebral fracture scores into a subject-level classification (e.g., prediction) of fracture. The classification may be performed, for example, by computer system 144 of FIG. 1 in accordance with the executable instructions for classifying 146. In some examples, the executable instructions for classifying 146 may include instructions for performing logistic regression (LR) and/or generalized additive models (GAM).

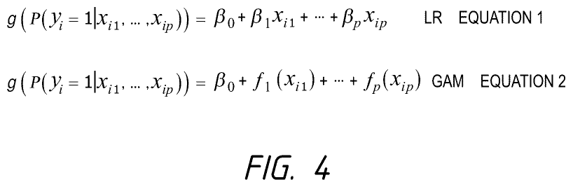

For each subject i and p inputs xᵢ₁ through xᵢₚ, the executable instructions for classifying 146 may include instructions for fitting a model of the form shown by Equations 1 and 2 in FIG. 4. For example, Equation 1 describes a form of model for LR. Equation 2 describes a form of model for GAM. Other forms of model may also be used and/or fit in accordance with executable instructions for classifying 146 of FIG. 1. In Equations 1 and 2 P(yᵢ=1|xᵢ₁, . . . , xᵢₚ) is the conditional probability of any vertebral fracture given the inputs, g(.) is the logit function, and f₁, . . . fₚ are nonlinear functions which may be estimated, for example, using scatterplot smoothers. LR may generally be a model used for binary outcomes. GAMs refer generally to an extension where the assumption that the log-odds are linear may be replaced by the more general form. By relaxing a linearity constraint, GAMs may generally increase model flexibility and capture nonlinearities in covariate effects. Regression models may advantageously be used in some examples due to the generally low dimensionality of the predictor space, which may avoid or reduce overfitting and facilitates understanding and ease of implementation in some examples.

For inputs, individual vertebral predictions or their order statistics may be provided as inputs for subject-level classification (e.g., the top k fracture predictions at the vertebral level). Order statistics like the maximum are nonlinear functions of the individual vertebral-level predictions. Interaction terms for neighboring anatomic levels may also be modeled, since fractures can be subtle and adjacent intermediate predictions should increase confidence of a fracture as multiple osteoporotic compression fractures typically are adjacent.

As described herein, clinical covariate data may be used together with scores from individual vertebral bodies to classify a radiograph of a spine. When working with medical imaging data, basic subject demographics may be embedded within the DICOM metadata of the file. Thus, subjects' age and gender data may be readily available. The computer system 144 may obtain clinical covariate data 126, for example, by accessing metadata associated with an image data file corresponding to a radiograph. The metadata may be stored, for example, in or together with image data 124.

In some examples, classification methods, such as those specified by the executable instructions for classifying 146 of FIG. 1 may weight vertebral scores from vertebrae in certain locations. In some examples, the executable instructions for classifying 146 may include instructions for selecting a representative score from each vertebral region (e.g., upper thoracic, mid thoracic, lower thoracic, thoraco-lumbar junction, and lower lumbar). In some examples, the representative score may be a maximum score of the individual VBs in that region. In some examples, the representative score may be an average score of the individual VBs in the region.

In some examples, interaction terms may be incorporated for neighboring vertebral pairs into a linear model. In an example, an LR model may utilize the top three maximum vertebral fracture scores including interaction terms between maxima.

Demographic covariates (e.g., age and race/ethnicity) may be incorporated into classifiers described herein. In both model classes of LR and GAM with all vertebrae as input, or in GAMs with multiple maximum vertebral body predictions, age may be added.

LR models and/or GAMs may be utilized using select combinations of individual vertebral facture scores and order statistics, as well as interaction terms between neighboring predictions (e.g., in rank or vertebral location). In this manner, a subject-level predictive model of moderate to severe fracture (e.g., osteoporotic vertebral fracture) may be created. In some examples, an indicator of the specific vertebral location of the maximum prediction and demographic covariates of age and race/ethnicity (e.g., from DICOM metadata) may be included.

Accordingly, while many systems may attempt or start by performing a task on a complex piece of data (e.g., entire radiograph, entire CT scan, etc.), examples described herein may function by breaking the task into smaller units (e.g., scoring and/or classification of individual VBs). Classification performance may be improved by classifying subcomponents and combining predictions for a clinical task. In some examples, taking the maximum predicted value may minimize or reduce error from individual predictions and facilitate explainable artificial intelligence (AI) results by knowing the region or regions of the radiograph responsible for the prediction of fracture.

Subject-level models may accordingly use all or selected scores from individual VBs. Classifier models may in some examples be based on the maximum vertebral fracture score, additional information specifying the location of the maximum decreased model performance, and/or additional order statistics.

In other examples, additional or different clinical covariate data may be used, such as structured or unstructured data sources including, but not limited to, electronic medical records or outputs from multiple models as a form of ensemble combination.

From the foregoing it will be appreciated that, although specific embodiments have been described herein for purposes of illustration, various modifications may be made while remaining within the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signals or communications may be provided directly to and/or received directly from the recited components without intervening components, but also may be provided to and/or received from the certain components through intervening components.

What is claimed is:

1. A method comprising:
extract, using a first trained machine learning model, image data portions corresponding to respective individual vertebral bodies from a two dimensional image of at least a portion of a spine;
input each of the image data portions to a second machine learning model to generate respective scores indicative of a likelihood each of the individual vertebral bodies has a fracture; and
classify the spine, using a subject-level classifier based on the scores, into a fracture status.

2. The method of claim 1 wherein the image data portions comprise first image data portions, and wherein the method further comprises:
extract, using a third trained machine learning model, second image data portions corresponding to the respective individual vertebral bodies from the two-dimensional image.

3. The method of claim 2, wherein the method further comprises combine an output of the first trained machine learning model and the third trained machine learning model to provide the image data portions input to the second machine learning model.

4. The method of claim 1, wherein said two-dimensional image is generated by an X-ray system.

5. The method of claim 1, wherein said classify the spine is further based on clinical covariate data.

6. The method of claim 5, wherein said clinical covariate data comprises body mass index (BMI).

7. The method of claim 1, further comprising display the fracture status overlaid on the two-dimensional image.

8. At least one computer readable media encoded with instructions, which, when executed, cause a system to perform operations comprising:
extract, using a first trained machine learning model, image data portions corresponding to respective individual vertebral bodies from a two-dimensional image of at least a portion of a spine;
input each of the image data portions to a second machine learning model to generate respective scores indicative of a likelihood each of the individual vertebral bodies has a fracture; and
classify the spine, using a subject-level classifier based on the scores, into a fracture status.

9. The at least one computer readable media of claim 8, wherein the image data portions comprise first image data portions, and wherein the operations further comprise:
extract, using a third trained machine learning model, second image data portions corresponding to the respective individual vertebral bodies from the two-dimensional image.

10. The at least one computer readable media of claim 9, wherein the operations further comprise combine an output of the first trained machine learning model and the third trained machine learning model to provide the image data portions input to the second machine learning model.

11. The at least one computer readable media of claim 8, wherein said two-dimensional image is generated by an X-ray system.

12. The at least one computer readable media of claim 8, wherein said classify the spine is further based on clinical covariate data.

13. The at least one computer readable media of claim 12, wherein said clinical covariate data comprises body mass index (BMI).

14. The at least one computer readable media of claim 8, wherein the operations further comprise display the fracture status overlaid on the two-dimensional image.

15. A system comprising:
at least one processor;
at least one computer readable media encoded with instructions which, when executed by the at least one processor, cause the system to perform operations comprising:
extract, using a first trained machine learning model, image data portions corresponding to respective individual vertebral bodies from a two-dimensional image of at least a portion of a spine;
input each of the image data portions to a second machine learning model to generate respective scores indicative of a likelihood each of the individual vertebral bodies has a fracture; and
classify the spine, using a subject-level classifier based on the scores, into a fracture status.

16. The system of claim 15, wherein the image data portions comprise first image data portions, and wherein the operations further comprise:
extract, using a third trained machine learning model, second image data portions corresponding to the respective individual vertebral bodies from the two-dimensional image.

17. The system of claim 16, wherein the operations further comprise combine an output of the first trained machine learning model and the third trained machine learning model to provide the image data portions input to the second machine learning model.

18. The system of claim 15, wherein said two-dimensional image is generated by an X-ray system.

19. The system of claim 15, wherein said classify the spine is further based on clinical covariate data.

20. The system of claim 19, wherein said clinical covariate data comprises body mass index (BMI).
